(19) United States
(12) Patent Application Publication
(10) Pub. No.: US 2006/0173555 A1
(43) Pub. Date: Aug. 3, 2006

(54) Elastic energy-stored artificial foot

(76) Inventors: Chuen-Sen Harn, Xin-Zhuang City (TW); Rueng-Jie Zhen, Xin-Zhuang City (TW)

Correspondence Address: RABIN & Berdo, PC, 1101 14TH STREET, NW, SUITE 500, WASHINGTON, DC, 20005, US

(21) Appl. No.: 11/047,702

(22) Filed: Feb. 2, 2005

(51) Int. Cl. A61F 2/66

(52) U.S. Cl. 623/52

(57) ABSTRACT

Disclosed is an elastic energy-stored artificial foot that effectively cushions vibration generated when walking and saves efforts through an energy-stored elasticity by the cushion when walking. The artificial foot includes a supporter supporting an artificial lower leg; a sole of an elastic material; a holder, an elastic plank downward bent to form a U-shaped structure of which an indent faces the back of the foot, and two slabs formed after the holder is bent are an upper connecting plank fixed to the supporter and a lower connecting plank fixed to the sole; an elastic part between the upper connecting plank and the lower connecting plank to provide a buffer capacity after the holder is suppressed; and a linking mechanism between the upper connecting plank and the lower connecting plank to hold a position of the holder compressed to transformation and prevent it from being shifted.

### BACKGROUND OF THE INVENTION

1. Field of the Invention

The present invention relates to an artificial foot, and particularly to an elastic energy-stored artificial foot that effectively cushions vibration induced by waking activity and provides an effort-saving performance through an energy-stored elasticity employed by the elastic cushion when walking.

2. The Related Art

Referring to FIG. 1, a conventional structure of artificial foot is formed with a body 1 copying a normal structure and function of a foot. The body 1 is equal to a human being's sole of the foot and may be placed into an artistically artificial foot 2 formed and constructed into a real foot. And the body 1 is provided with a supporter 3 to connect with an artificial leg so that amputees may recover an original function of the foot they had using the support of their artificial feet. Consequently, in order to have the structure of artificial foot work and be close to the bones and skeleton of leg and to the muscle of the human being that are cooperative to make the user easily go on foot and to prevent the user from tripping, an inventor once improved an almost immovable structure of an artificial foot and added to the artificial foot a structure that is similar to a normal ankle and provides a function making the sole of the foot move forward, backward, upward, and downward, in which a universal bearing is used to have the artificial foot based on the ankle as a kernel move forward, backward, leftward, and rightward according to landforms so as to walk on a road comparative steep.

However, this structure that is based on the bearing is complicated in construction, and is thus uneconomical in manufacturing cost and time.

Then, a forepaw and a heel of the artificial foot are respectively provided with a gasbag as a member to which a rib is pressed and fixed, or front and rear elastic parts are used working with a revolving part or the bearing at the ankle so as to make the sole of artificial foot or the heel turn frontward and backward according to the landforms. However, the conventional structure provides only the function making the artificial foot wiggle frontward and backward according to the landforms, but a reacting force caused by a footstep on the ground was not considered so that the user feels uncomfortable due to the user's limbs connected to an artificial limb sheath.

Thus, an ankle block 4 with condensable foam (as shown in FIG. 1) is used as a structural part of the ankle area to absorb the shock and turn. However, although this conventional technique solves the defects described above, the entire foam is an elastic part and it not only evenly presses or stretches from the foam near a front end of the sole of foot or a back end of the heel, when the pressure caused by the footstep on the ground occurs, and may also lay a pressure at an inclined angle so that an upper end connected to an artificial lower leg is formed with an inclined outward force and thereby the user walks unstably and trips over easily.

### SUMMARY OF THE INVENTION

In order to provide a proper capability of absorbing a shock to an artificial foot, make a user comfortably walk through an energy-stored elasticity, and meanwhile prevent the user's footstep from being unstable or the user from tripping due to a cushion structure suppressed and displaced, the present invention provides an elastic energy-stored artificial foot that may effectively absorbs a shock caused when walking and may provide an effort-saving performance through a energy-stored elasticity caused by an elastic cushion when walking; also, a linking mechanism is used to prevent the cushion from being displaced for making comfort ability and safety available.

The present invention comprises a supporter supporting an artificial lower leg; a sole made of an elastic material, of which a shape and size is based on an artistic artificial foot to be placed; a holder, namely an elastic plank downward bent to form a U-shaped structure of which an indent faces the back of energy-stored foot, in which two slabs formed after the holder is bent are respectively an upper connecting plank fixed to the supporter and a lower connecting plank fixed to the sole; an elastic part located between the upper connecting plank and the lower connecting plank to provide a buffer capacity after the holder is suppressed; and a linking mechanism located between the upper connecting plank and the lower connecting plank. Both the sole and the holder according to the present invention may be made of a light and elastic carbon fiber of stress, and the holder may be structured with three layers of carbon-fiber planks. Besides, the linking mechanism may further comprise an upper link seat, two first links, two second links, and a lower link seat. The upper and lower link seats are respectively located on the upper and lower connecting planks and orderly pivotally connected from the upper link seat to the first links, the second links, and the upper link seat in a space between the upper and lower connecting planks, and thereby the linking mechanism provided with a transformed position keeping the holder is formed. When the user walks on the elastic energy-stored foot, the pressure from the supporter is downward transmitted through the elastic holder and meanwhile provides a downward buffer capacity by means of the elastic element for a comfortable walking. On the other hand, the elastic element after suppressed compresses the stored elasticity, so the foot is lifted saving more efforts, and through an operation of the linking mechanism complying with the holder and the elastic element, a downward pressure is balanced at both sides so that a direction of the transformation of holder cannot be displaced due to an inclination toward the left and right sides, thereby a stability of footsteps on the march being increasing and cases of tripping being decreasing.

### BRIEF DESCRIPTION OF THE DRAWINGS

FIG. 1 is a schematic view illustrating a conventional artificial foot.

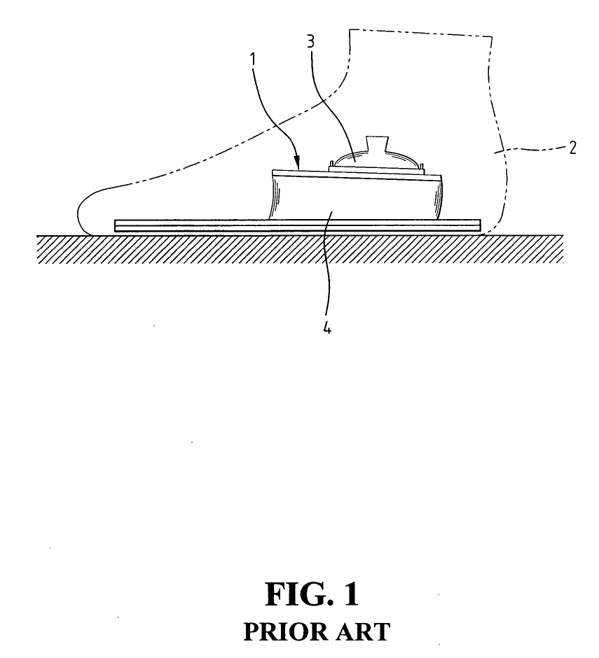

FIG. 2 is a side view of an artificial foot constructed in accordance with the present invention.

FIG. 3 is a perspective view of the artificial foot of the present invention.

FIG. 4 is an exploded view of the artificial foot of the present invention.

FIG. 5 is an exploded view of a linking mechanism in accordance with the present invention.

FIG. 6 is a side elevational view, illustrating operation of the artificial foot of the present invention.

### DETAILED DESCRIPTION OF THE PREFERRED EMBODIMENTS

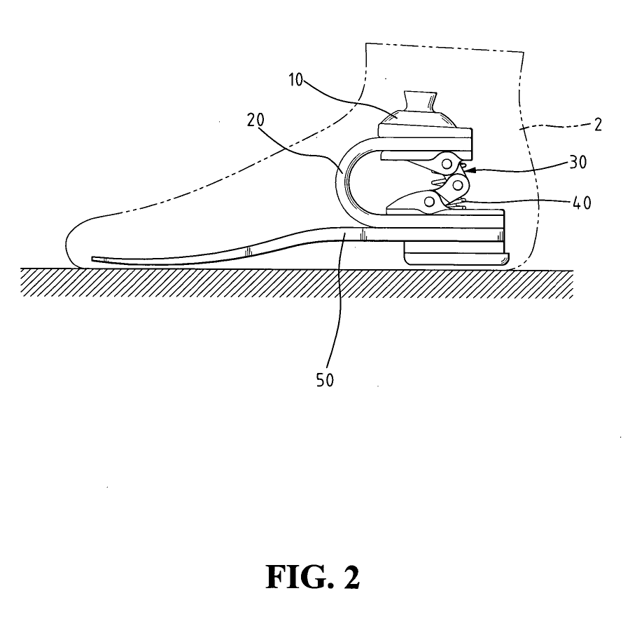

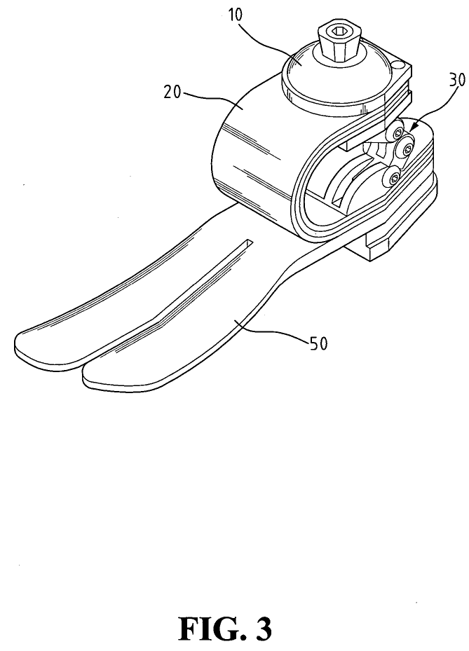

FIGS. 2 and 3 illustrate an artificial foot, designated with reference numeral 2, constructed in accordance with the present invention. The artificial foot 2 comprises a supporter 10, a holder 20, a linking mechanism 30, an elastic element 40, and a sole 50. A top of the holder 20 is connected with the supporter 10 and a bottom of the holder 20 is fixed with the sole 50. The linking mechanism 30 is provided at a U-shaped space formed by the holder 20. In the embodiment according to the present invention, a real structure of foot is integrally formed, and the holder 20, the elastic element 40, and the linking mechanism 30 are structured into an elastic structure of cushion similar to an ankle in function. In the embodiment according to the present invention, the supporter 10 is upwardly connected to an artificial leg and jointed to a fixing end located therein and the rest are placed into an artistic artificial foot copying features of a real foot so that amputees may have the artificial foots according to the present invention connected with the artificial legs and then may recover to walk on their limbs.

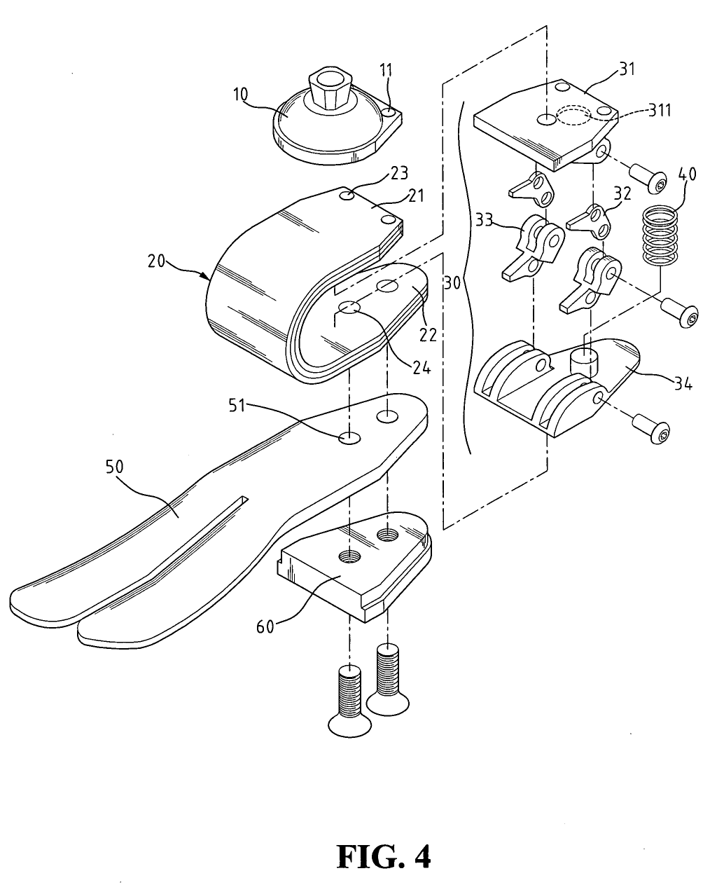

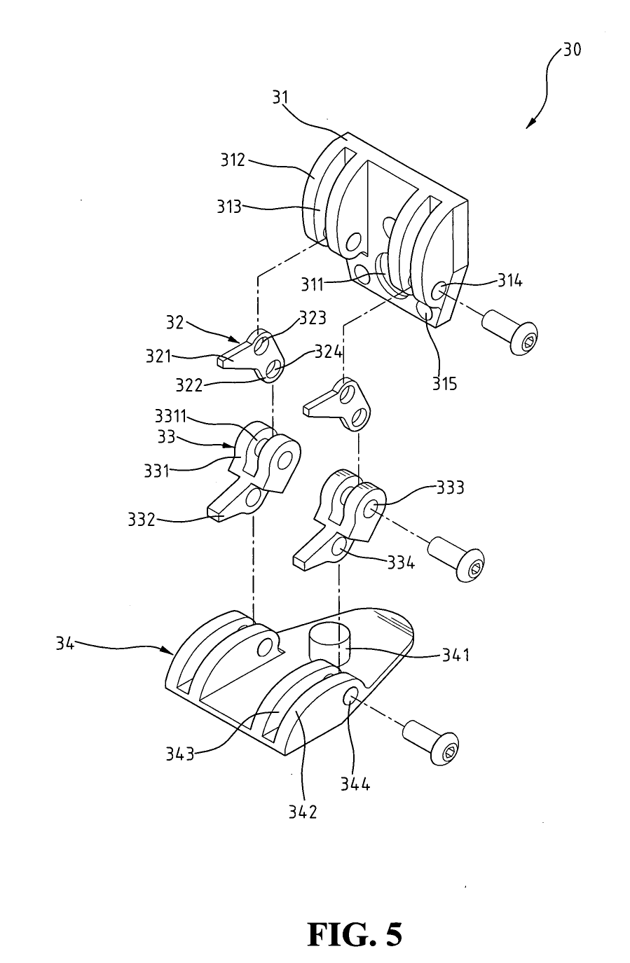

Referring to FIGS. 4 and 5, the holder 20 is a flexible elastic slab and downward bendable to form a horizontal U-shaped structure of which an indent faces the back of energy-stored foot, and after the holder 20 but an arc area is bent, two slabs are formed and are respectively an upper connecting plank 21, fixed to the supporter 10 through a thru hole 23 provided, and a lower connecting plank, fixed to the sole 50 through a thru hole 24 provided. The holder 20 is a flexible, elastic material that may be a material of carbon fiber, elastic rubber, and the like. If the holder 20 is the material of carbon fiber, it may be structured with three layers of carbon-fiber planks for a better effect of elastic cushion.

The linking mechanism 30 comprises an upper link seat 31, two first links 32, two second links 33, and a lower link seat 34. The upper link seat 31 is provided with a thru hole 315, and through the thru hole 23 and a thru hole 11, the upper link seat 31 may be fixed to a bottom of the upper connecting plank 21 and together to the supporter 10. The upper link seat 31 is provided with two first pivot-joint lugs 312 provided with a first pivot-joint wedging slot 313 and a pivot-joint hole 314; the first links 32 are respectively provided with a first pivot-joint terminal 321 and a second pivot-joint terminal 322, in which the first pivot-joint terminal 321 is provided with a first pivot-joint hole 323 corresponding to the pivot-joint hole 314 and the second pivot-joint terminal is provided with a first pivot-joint hole 324 corresponding to a pivot-joint hole 333; the second links 33 are also respectively provided with a third pivot-joint terminal 331 and a fourth pivot-joint terminal 332, in which the third pivot-joint terminal 331 is provided with a second pivot-joint wedging slot 3311; the lower link seat 34 may be fixed to a top of the lower connecting plank 22 and it is provided with two second pivot-joint lug 342 on which a third pivot-joint wedging slot 343 is provided.

When the linking mechanism 30 in the embodiment according to the present invention is installed, the first pivot-joint terminal 321 may be in advance wedged into the first pivot-joint wedging slot 313 and may be pivotally connected to the first pivot-joint lug 312 through the pivot-joint hole 314 and the first pivot-joint hole 323, the second pivot-joint terminal 322 is wedged into the second pivot-joint wedging slot 3311 and pivotally connected to the third pivot-joint terminal 331 through the pivot-joint hole 324 and the first pivot-joint hole 333, and the fourth pivot-joint terminal 332 is wedged into the third pivot-joint wedging slot 343 and pivotally connected to the second pivot-joint lug 342 through the pivot-joint hole 334 and the first pivot-joint hole 344; thereby, the linking mechanism orderly connected to the first link 32, the second link 33, and finally the lower link seat 34 from the upper link seat 31 is formed.

On the other hand, the elastic element 40 may be located between the upper connecting plank 21 and the lower connecting plank 22 for achievement of the performance of buffer when the holder 20 is suppressed. In the embodiment according to the present invention, the upper link seat 31 and the lower link seat 34 are respectively fixed to the upper connecting plank 21 and the lower connecting plank 22, so the elastic element 40 may also be located between the upper link seat 31 and the link seat 34. The elastic element 40 may be a spring or a cushion of elasticity, and it is made of a material having a supporting capability, such as high-density bubble polyurethane foam. When the elastic element 40 works as the spring, a concave hole 311 may be provided in the upper link seat 31 and a protruding pillar 341 is extensionally provided in the lower link seat 34; then, a top of the spring is inserted into the concave hole 311 and an end tip of the spring is put around the pillar 341 so that the spring may be fixed between the upper link seat 31 and the lower link seat 34, and thereby when the upper connecting plank 21 is downward pressed, a buffer capability is provided for keeping the foot steps stable, and through the compression of spring, a proper elasticity is stored and thereby the foot is easily lifted saving more efforts. Further, the sole 50 is made of an elastic material, of which a shape and size is based on an artistic artificial foot to be placed, and the material may be of carbon fiber or the like similar. A sole base 50 may be further provided under the sole to enhance the stability of elastic energy-stored foot.

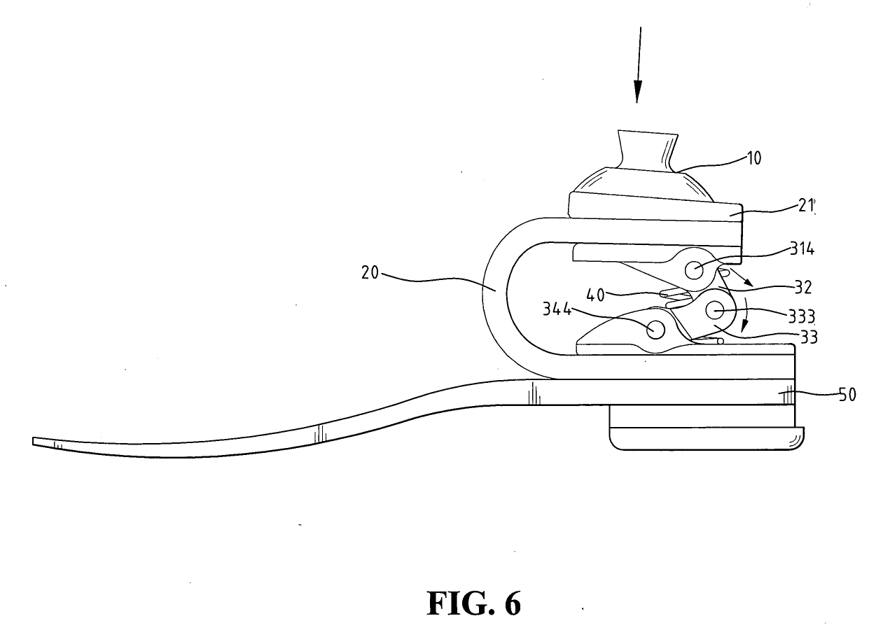

Referring now to FIG. 6, when the user walks on foot, a downward pressure comes from the supporter 10 and the downward pressure may have the sole 50 of elastic stress transformed due to the buffer, and also the elastic holder 20 at the upper connecting plank 21 is downward bent; the transformation has the elastic element 40 compressed and at the same time coordinates with the linking mechanism 30 to work together. At the time of coordination, the first link 32 pivots on the pivot-joint hole 314 and moves obliquely backward and downward, while the second link 33 pivots on the pivot-joint hole 344 and shifts downward, and thereby the linking mechanism completely links due to the compression. Because the links are installed at right and left sides and a section of the original holder 20 bent forms a quadrilateral plane surface, even if downward pressures applied at right and left sides are uneven, the upper connecting plank 21 is made to evenly shift to the right and left sides but not to be at the state of right and left excursion, thereby the stability of walk on foot being greatly enhanced and the cases of tripping being decreased.

What is claimed is:

1. An elastic energy-stored artificial foot comprising:
a supporter adapted to support an artificial lower leg;
a sole made of an elastic material;
a holder serving as an elastic plank downward bent to form a U-shaped structure of which an indent faces the back of energy-stored foot, in which two slabs formed after the holder is bent are respectively an upper connecting plank fixed to the supporter and a lower connecting plank fixed to the sole;
an elastic element located between the upper connecting plank and the lower connecting plank to provide a buffer capacity after the holder is suppressed; and
a linking mechanism located between the upper connecting plank and the lower connecting plank in order to keep a position of the holder compressed to transformation and to prevent it from being shifted.

2. The elastic energy-stored artificial foot as claimed in claim 1, wherein the sole is made of carbon fiber.

3. The elastic energy-stored artificial foot as claimed in claim 1, wherein the holder is made of the carbon fiber.

4. The elastic energy-stored artificial foot as claimed in claim 3, wherein the holder comprises three layers of carbon-fiber planks.

5. The elastic energy-stored artificial foot as claimed in claim 1, wherein the elastic element comprises a spring.

6. The elastic energy-stored artificial foot as claimed in claim 1, wherein the elastic element comprises high-density bubble polyurethane foam.

7. The elastic energy-stored artificial foot as claimed in claim 1, wherein the linking mechanism comprises:
an upper link seat fixed to a bottom of the upper connecting plank and provided with two first pivot-joint lugs provided with a first pivot-joint wedging slot;
two first links respectively provided with a first pivot-joint terminal and a second pivot-joint terminal;
two second links respectively provided with a third pivot-joint terminal and a fourth pivot-joint terminal, in which the third pivot-joint terminal is provided with a second pivot-joint wedging slot; and
a lower link seat fixed to a top of the lower connecting plank and provided with two second pivot-joint lugs provided with a third pivot-joint wedging slot;
the first pivot-joint terminal being wedged into the first pivot-joint wedging slot and pivotally connected to the first pivot-joint lug; the second pivot-joint terminal being wedged into the second pivot-joint wedging slot and pivotally connected to the third pivot-joint terminal; the fourth pivot-joint terminal being wedged into the third pivot-joint wedging slot and pivotally connected to the second pivot-joint lug; thereby, the linking mechanism orderly connected to the first link, the second link, and finally the lower link seat from the upper link seat being formed.

8. The elastic energy-stored artificial foot as claimed in claim 1, wherein a sole base is further provided under the sole to enhance the stability of elastic energy-stored foot.
